(19) United States
(12) Patent Application Publication
(10) Pub. No.: US 2023/0100337 A1
(43) Pub. Date: Mar. 30, 2023

(54) DEVICE AND METHOD FOR DETECTING MOTION OF A SURFACE

(71) Applicant: PHOTONO OY, HELSINKI (FI)

(72) Inventors: Antti KONTIOLA, HELSINKI (FI); Patrick GRAHN, HELSINKI (FI)

(21) Appl. No.: 17/798,843

(22) PCT Filed: Dec. 17, 2020
(86) PCT No.: PCT/FI2020/050844

§ 371 Date: Aug. 10, 2022

(30) Foreign Application Priority Data
Feb. 11, 2020 (FI) 20205141

(51) Int. Cl. A61B 3/16

(52) U.S. Cl. CPC A61B 3/165

(57) ABSTRACT

A device for detecting motion of a surface includes a light source for emitting light, a focusing lens configured to focus the light, and a detector configured to receive the focused light reflected off the surface and to detect motion of a distribution pattern of the reflected light. The motion of the distribution pattern of the reflected light is indicative of the motion of the surface. A conic constant of the focusing lens is in the range from □1.5 to □0.5, and a diameter of the focusing lens is at least 60% of the distance from the focusing lens to a beam waist of the focused light. When the conic constant and the diameter are within the above-mentioned ranges, the device is suitable for handheld apparatuses for free-hand measurements of small motions of surfaces. A handheld apparatus can be for example an apparatus for detecting eye pressure.

### TECHNICAL FIELD

The disclosure relates to a device for detecting motion of a surface, for example a wave motion occurring on a surface of an eye. Furthermore, the disclosure relates to a method for detecting motion of a surface. Furthermore, the disclosure relates to an apparatus for detecting pressure of an eye.

### BACKGROUND

In many cases, there is a need to detect motion of a surface. For example, detection of surface motion can be utilized in eye pressure measurements where an airborne excitation such as e.g. an air pressure pulse, an ultrasonic tone burst, a shock wave, or some other suitable airborne excitation is used to deform a surface of an eye and thereafter an estimate of the eye pressure is obtained based on motion caused by the excitation on the surface of the eye. Motion of a surface can be detected for example with a detector that is configured to receive light reflected off the surface and to detect motion of a distribution pattern of the received light. The motion of the distribution pattern of the received light is indicative of the motion of the surface that has reflected the light.

In many applications, the above-described technique for detecting motion of a surface is however not free from challenges. For example, in conjunction with eye pressure measurements, it can be challenging to keep an excitation device that directs an airborne excitation to a surface of an eye and a detector device that detects motion of the surface of the eye sufficiently stationary with respect to the eye so that unintentional changes in the position and/or orientation of the excitation and detector devices with respect to the eye do not disturb the eye pressure measurement too much. The above-described challenge is present especially in conjunction with handheld tonometers and other handheld apparatuses which are used with a free-hand. Thus, there is a need for technical solutions for robust freehand measurements of motions of surfaces.

### SUMMARY

The following presents a simplified summary to provide basic understanding of some aspects of different invention embodiments. The summary is not an extensive overview of the invention. It is neither intended to identify key or critical elements of the invention nor to delineate the scope of the invention. The following summary merely presents some concepts of the invention in a simplified form as a prelude to a more detailed description of exemplifying and non-limiting embodiments of the invention.

In this document, the word “geometric” when used as a prefix means a geometric concept that is not necessarily a part of any physical object. The geometric concept can be for example a geometric point, a straight or curved geometric line, a geometric plane, a non-planar geometric surface, a geometric space, or any other geometric entity that is zero, one, two, or three dimensional.

In accordance with the invention, there is provided a new device for detecting motion of a surface. A device according to the invention comprises:
a light source configured to emit light,
a focusing lens configured to focus the light, and
a detector configured to receive the focused light reflected off the surface and to detect motion of a distribution pattern of the received light, the motion of the distribution pattern of the received light being indicative of the motion of the surface reflecting the light.

The focusing lens is an aspherical lens having a conic constant in the range from -1.5 to -0.5, and a diameter of the focusing lens is at least 60% of a distance from a light egress surface of the focusing lens to a beam waist of the focused light.

The above-mentioned range of the conic constant corresponds to spherical aberration which means that light beams at different distances from an optical axis of the focusing lens are focused at different distances from the focusing lens, and therefore the beam waist is lengthened in a direction of the optical axis, but on the other hand, the beam waist is widened in directions perpendicular to the optical axis. The beam waist is however narrow enough for motion detection, but the increased length of the beam waist provides more robustness against non-optimal position and/or orientation of the device with respect to a surface whose motion is being detected. When the beam waist of the focused light is at least near to a curved surface e.g. a surface of an eye, the light reflected off the curved surface has the following two advantageous properties: i) a wide angular distribution and ii) a power distribution whose maximum is in a direction of a specular reflection and whose shape is advantageous for detecting movement of a light spot formed by the reflected light on an imaging plane that is perpendicular to the direction of the specular reflection. Thanks to the above-mentioned wide angular distribution, a sufficient part of the reflected light can be collected to the detector even if the beam waist is not exactly at the curved surface but only near to the curved surface. The combination of the above-mentioned two advantageous properties enables robust freehand measurements of small motions of surfaces.

In accordance with the invention, there is provided also a new apparatus for detecting pressure of an eye. The measured pressure is typically the intraocular pressure “IOP” of the eye. An apparatus according to the invention comprises:
an excitation source configured to direct airborne excitation, e.g. an air pressure pulse, an ultrasonic tone burst, or a shock wave, to the eye to deform a surface of the eye,
a device according to the invention and configured to detect motion of the surface of the eye, and
a processing device configured to determine an estimate of the pressure of the eye based on the detected motion of the surface of the eye.

In accordance with the invention, there is provided also a new method for detecting motion of a surface. A method according to the invention comprises:
emitting light,
focusing the light with a focusing lens so that the focused light is directed towards the surface, and
detecting motion of a distribution pattern of the focused light reflected off the surface, the motion of the distribution pattern of the reflected light being indicative of the motion of the surface,

wherein the focusing lens is an aspherical lens having a conic constant in the range from -1.5 to -0.5, and a diameter of the focusing lens is at least 60% of a distance from a light egress surface of the focusing lens to a beam waist of the focused light.

Various exemplifying and non-limiting embodiments are described in accompanied dependent claims.

Exemplifying and non-limiting embodiments both as to constructions and to methods of operation, together with additional objects and advantages thereof, are best understood from the following description of specific exemplifying embodiments when read in conjunction with the accompanying drawings.

The verbs “to comprise” and “to include” are used in this document as open limitations that neither exclude nor require the existence of un-recited features. The features recited in dependent claims are mutually freely combinable unless otherwise explicitly stated. Furthermore, it is to be understood that the use of “a” or “an”, i.e. a singular form, throughout this document does not exclude a plurality.

### BRIEF DESCRIPTION OF FIGURES

Exemplifying and non-limiting embodiments and their advantages are explained in greater detail below with reference to the accompanying drawings, in which:

FIG. 1 illustrates a device according to an exemplifying and non-limiting embodiment for detecting motion of a surface,

FIGS. 2a, 2b, and 2c illustrate focusing lenses of devices according to exemplifying and non-limiting embodiments for detecting motion of a surface,

FIG. 3 illustrates an apparatus according to an exemplifying and non-limiting embodiment for detecting pressure of an eye, and

FIG. 4 shows a flowchart of a method according to an exemplifying and non-limiting embodiment for detecting motion of a surface.

### DESCRIPTION OF EXEMPLIFYING AND NON-LIMITING EMBODIMENTS

The specific examples provided in the description below should not be construed as limiting the scope and/or the applicability of the accompanied claims. Lists and groups of examples provided in the description below are not exhaustive unless otherwise explicitly stated.

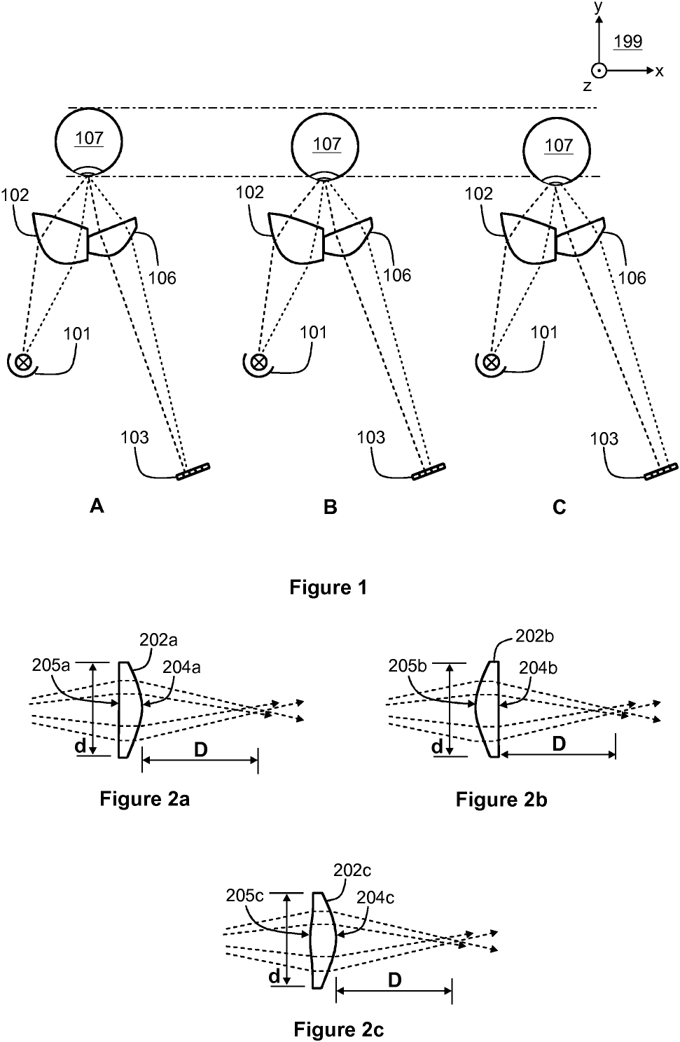

FIG. 1 illustrates a device according to an exemplifying and non-limiting embodiment for detecting motion of a surface. Operation of the device for detecting motion of a surface of an eye 107 is illustrated in three different exemplifying situations A, B, and C. In the exemplifying situation A, the eye 107 is at a given position with respect to the device. In the exemplifying situation B, the eye 107 has been shifted in the negative y-direction of a coordinate system 199 with respect to the exemplifying situation A. In the exemplifying situation C, the eye 107 has been further shifted in the negative y-direction of the coordinate system 199 with respect to the exemplifying situation B.

The device comprises a light source 101 for emitting light. The light source 101 may comprise for example one or more light emitting diodes “LED”, one or more laser diodes, one or more filament lamps, one or more gas discharge lamps, or some other suitable light emitting element or elements. The device comprises a focusing lens 102 configured to focus the light. The focusing lens 102 is an aspherical lens having a conic constant in the range from -1.5 to -0.5. More advantageously, the conic constant is in the range from -1.3 to -0.6. Yet more advantageously, the conic constant is in the range from -1.1 to -0.7. Even more advantageously, the conic constant is in the range from -0.9 to -0.7. A diameter of the focusing lens 102 is at least 60% of a distance from a light egress surface of the focusing lens to a beam waist of the focused light. More advantageously, the diameter is at least 70% of the distance between the beam waist and the light egress surface. Yet more advantageously, the diameter is at least 90% of the above-mentioned distance. Even more advantageously, the diameter is at least 110% of the distance i.e. the diameter is at least 1.1 × the distance. The diameter of the focusing lens 102 is at most 400% of the distance i.e. the diameter is at most 4 × the distance. When the conic constant and the diameter are within the above-mentioned ranges, the device is suitable for free-hand measurements of small motions of surfaces. In cases where the focusing lens is not circular when seen along an optical axis of the focusing lens, the diameter of the focusing lens is a diameter of a greatest geometric circle that can be inside outlines of the focusing lens when seen along the optical axis.

The device comprises a detector 103 configured to receive the focused light reflected off the surface of the eye 107 and to detect motion of a distribution pattern of the received light. The motion of the distribution pattern of the received light is indicative of the motion of the surface of the eye 107. The exemplifying device illustrated in FIG. 1 comprises a collector lens 106 for directing, to the detector 103, the light reflected off the surface of the eye 107. The detector 103 may comprise for example an array of photosensor elements such as e.g. photodiodes or phototransistors. In this exemplifying case, the detector 103 can be configured to operate as a differential sensor where changes in differences between output signals of the photosensor elements are indicative of the motion of the distribution pattern of the received light. It is also possible that the detector 103 comprises e.g. a charge coupled device “CCD” or some other suitable sensor for detecting motion of the distribution pattern of the received light.

FIGS. 2a, 2b, and 2c illustrate focusing lenses 202a, 202b, and 202c of devices according to exemplifying and non-limiting embodiments for detecting motion of a surface. FIG. 2a shows an exemplifying case where the focusing lens 202a is a plano-convex lens so that a light egress surface 204a of the focusing lens has the conic constant in the range from -1.5 to -0.5 and a light ingress surface 205a of the focusing lens is planar. FIG. 2b shows an exemplifying case where the focusing lens 202b is a plano-convex lens so that a light ingress surface 205b of the focusing lens has the conic constant in the range from -1.5 to -0.5 and a light egress surface 204b of the focusing lens is planar. FIG. 2c shows an exemplifying case where a light egress surface 204c of the focusing lens 202c is non-planar and a light ingress surface 205c of the focusing lens 202c is non-planar so that a combined optical effect of the light egress and light ingress surfaces 204c and 205c is the same as an optical effect of a plano-convex lens whose conic constant of a convex surface is in the range from -1.5 to -0.5. In each of FIGS. 2a, 2b, and 2c, the distance from the light egress surface to a beam waist of focused light is denoted with D and the diameter of the focusing lens is denoted with d.

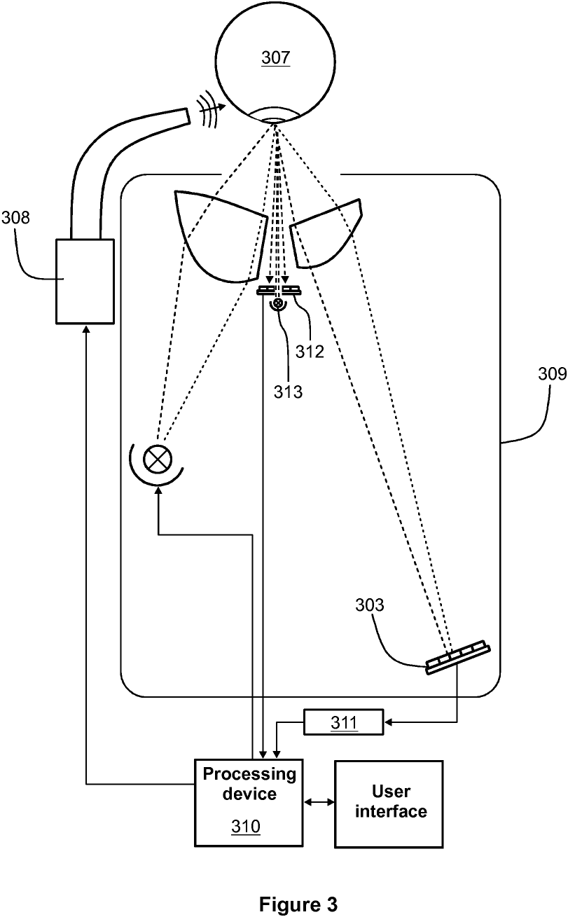

FIG. 3 illustrates an apparatus according to an exemplifying and non-limiting embodiment for detecting pressure of an eye 307. The apparatus comprises an excitation source 308 configured to direct airborne excitation to the eye 307 to deform a surface of the eye. The airborne excitation can be e.g. an air pressure pulse, an ultrasonic tone burst, or a shock wave. The apparatus comprises a detector device 309 for detecting motion of the surface of the eye 307. The detector device 309 can be for example such as the device illustrated in FIG. 1. The apparatus comprises a processing device 310 configured to determine an estimate of the pressure of the eye 307 based on the detected motion of the surface of the eye.

In an apparatus according to an exemplifying and non-limiting embodiment, the processing device 310 is configured to measure time between a first time instant when the excitation source 308 directs the airborne excitation to a first spot on the surface of the eye 307 and a second time instant when the detector device 309 detects motion from a second spot on the surface of the eye 307. The processing device 310 is configured to determine the estimate of the pressure of the eye based on the measured time. The speed of waves propagating on the surface of the eye 307 depends on the pressure of the eye. Therefore, the above-mentioned measured time, i.e. a ‘time-of-flight’, is indicative of the pressure of the eye.

In an apparatus according to an exemplifying and non-limiting embodiment, the processing device 310 is configured to measure oscillation frequency related to the motion of the surface of the eye 307, and to determine the estimate of the pressure of the eye based on the measured oscillation frequency. In this exemplifying case, the eye pressure measurement is based on the fact that oscillation frequency of a displacement in a direction perpendicular to the surface of the eye 307 depends on the pressure of the eye.

In an apparatus according to an exemplifying and non-limiting embodiment, the processing device 310 is configured to correct the estimate of the pressure of the eye 307 in accordance with a predetermined correction rule based on a location and/or a size of a light spot on a light receiving area of the detector 303. The location and/or the size of the light spot is indicative of a position and an orientation of the apparatus with respect to the eye 307. Thus, a non-optimal position and/or orientation of the apparatus with respect to the eye 307 can be detected based on the location and/or the size of the light spot on the light receiving area of the detector 303. Referring to FIG. 1, we can assume for example that the exemplifying situation A corresponds to an optimal position of the device illustrated in FIG. 1 with respect to the eye 107 and that the exemplifying situations B and C correspond to non-optimal positions of the device with respect to the eye 107. As illustrated in FIG. 1, the location and/or the size of the light spot on the light receiving area of the detector 103 are/is different in different ones of the exemplifying situations A, B, and C. The above-mentioned correction rule can be e.g. a lookup table based on empirical test results which indicate an effect of non-optimal positions and/or orientations on the pressure estimate. It is also possible that above-mentioned correction rule is a parametrized mathematical formula whose parameters are based on empirical test results of the kind mentioned above.

In an exemplifying and non-limiting embodiment, the apparatus further comprises a photosensor array 312 configured to receive light reflected off the eye 307. The processing device 310 is configured to correct the estimate of the pressure of the eye in accordance with a predetermined rule based on a position of a pattern of the received light on the photosensor array 312. The photosensor array 312 can be for example an array of photodiodes or phototransistors. In this exemplifying case, the apparatus may further comprise a light source 313 e.g. a laser diode, a LED, or some other suitable light emitting element. In a case where the apparatus is non-optimally positioned and/or oriented with respect to the eye 307, the position of the pattern of the received light on the photosensor array 312 deviates from a position corresponding to an optimal position and orientation of the apparatus with respect to the eye 307. This deviation can be used for correcting the estimate of the pressure of the eye. The predetermined rule can be e.g. a lookup table based on empirical test results which indicate an effect of non-optimal positions and/or orientations on the pressure estimate. It is also possible that above-mentioned predetermined rule is a parametrized mathematical formula whose parameters are based on empirical test results of the kind mentioned above. In an exemplifying and non-limiting embodiment, the photosensor array 312 is a photosensor array of a digital camera. In this exemplifying case, the photosensor array 312 can be e.g. a charge coupled device “CCD” or some other suitable camera element. The digital camera may operate with the aid of ambient light and/or a part of the light directed to the eye 307 by the focusing lens. It is also possible that the apparatus comprises a separate light source e.g. a laser diode or a LED for producing at least a part of the light received by the digital camera. In a case where the apparatus is non-optimally positioned and/or oriented with respect to the eye 307, an image produced by the digital camera i.e. a position and/or a shape of the pattern of the received light on the photosensor array 312 deviates from an image corresponding to an optimal position and orientation of the apparatus with respect to the eye 307. This deviation can be detected by image recognition and it can be used for correcting the estimate of the pressure of the eye.

An apparatus according to an exemplifying and non-limiting embodiment comprises a signal processing element 311 for processing an output signal or output signals of the detector 303. The signal processing element 311 may comprise for example a filter that attenuates undesirable frequency components of the output signal or signals of the detector 303.

The processing device 310 can be implemented with one or more processor circuits, each of which can be a programmable processor circuit provided with appropriate software, a dedicated hardware processor such as for example an application specific integrated circuit “ASIC”, or a configurable hardware processor such as for example a field programmable gate array “FPGA”. The software may comprise e.g. firmware that is a specific class of computer software that provides low-level control for hardware of the processing device 310. The firmware can be e.g. open-source software. Furthermore, the processing device 310 may comprise one or more memory circuits each of which can be for example a random-access-memory “RAM” circuit.

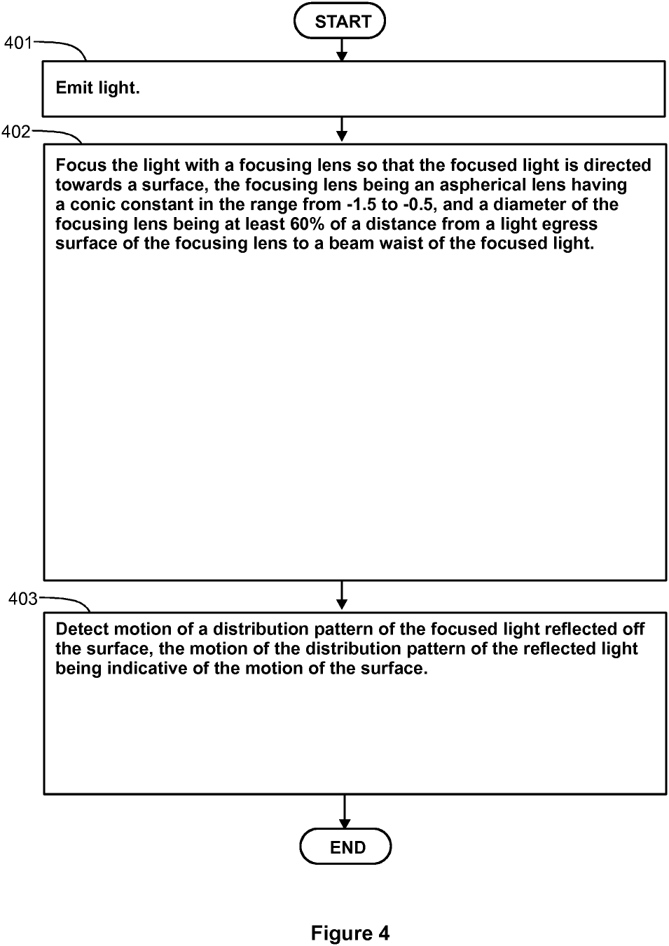

FIG. 4 shows a flowchart of a method according to an exemplifying and non-limiting embodiment for detecting motion of a surface. The method comprises the following actions:
action 401: emitting light,
action 402: focusing the light with a focusing lens so that the focused light is directed towards the surface, the focusing lens being an aspherical lens having a conic constant in the range from -1.5 to -0.5, and a diameter of the focusing lens being at least 60% of a distance from a light egress surface of the focusing lens to a beam waist of the focused light, and
action 403: detecting motion of a distribution pattern of the focused light reflected off the surface, the motion of the distribution pattern of the reflected light being indicative of the motion of the surface.

In a method according to an exemplifying and non-limiting embodiment, the conic constant is in the range from -1.3 to -0.6.

In a method according to an exemplifying and non-limiting embodiment, the conic constant is in the range from -1.1 to -0.7.

In a method according to an exemplifying and non-limiting embodiment, the conic constant is in the range from -0.9 to -0.7.

In a method according to an exemplifying and non-limiting embodiment, the diameter of the focusing lens is at least 70% of the distance from the light egress surface of the focusing lens to the beam waist of the focused light.

In a method according to an exemplifying and non-limiting embodiment, the diameter of the focusing lens is at least 90% of the distance from the light egress surface of the focusing lens to the beam waist of the focused light.

In a method according to an exemplifying and non-limiting embodiment, the diameter of the focusing lens is at least 110% of the distance from the light egress surface of the focusing lens to the beam waist of the focused light.

In a method according to an exemplifying and non-limiting embodiment, the focusing lens is a plano-convex lens so that the light egress surface of the focusing lens has the conic constant in the range from -1.5 to -0.5 and the light ingress surface of the focusing lens is planar.

In a method according to an exemplifying and non-limiting embodiment, the focusing lens is a plano-convex lens so that the light ingress surface of the focusing lens has the conic constant in the range from -1.5 to -0.5 and the light egress surface of the focusing lens is planar.

In a method according to an exemplifying and non-limiting embodiment, the light egress surface of the focusing lens is non-planar and the light ingress surface of the focusing lens is non-planar so that a combined optical effect of the light ingress and light egress surfaces is the same as an optical effect of a plano-convex lens whose conic constant of a convex surface is in the range from -1.5 to -0.5.

In a method according to an exemplifying and non-limiting embodiment, the motion of the distribution pattern of the reflected light is detected with an array of photosensor elements. The detection can be based on changes in differences between output signals of the photosensor elements because these changes are indicative of the motion of the distribution pattern of the reflected light.

In a method according to an exemplifying and non-limiting embodiment, the light reflected off the surface is directed to a detector with the aid of a collector lens.

An eye pressure measurement method according to an exemplifying and non-limiting embodiment comprises the following actions:
directing airborne excitation to an eye to deform a surface of the eye,
carrying out a method according to an embodiment of the invention for detecting motion of the surface of the eye, and
determining an estimate of pressure of the eye based on the detected motion of the surface of the eye.

An eye pressure measurement method according to an exemplifying and non-limiting embodiment comprises measuring time between a first time instant when the airborne excitation is directed to a first spot on the surface of the eye and a second time instant when the motion is detected from a second spot on the surface of the eye, and determining the estimate of the pressure of the eye based on the measured time.

An eye pressure measurement method according to an exemplifying and non-limiting embodiment comprises measuring oscillation frequency related to the motion of the surface of the eye and determining the estimate of the pressure of the eye based on the measured oscillation frequency.

An eye pressure measurement method according to an exemplifying and non-limiting embodiment comprises correcting the estimate of the pressure of the eye in accordance with a predetermined correction rule based on a location and/or a size of a light spot on a light receiving area of a detector. The location and/or the size of the light spot is indicative of a position and an orientation of a measuring apparatus with respect to the eye. Thus, a non-optimal position and/or orientation of the measuring apparatus with respect to the eye can be detected based on the location and/or the size of the light spot on the light receiving area of the detector. The above-mentioned correction rule can be e.g. a lookup table based on empirical test results which indicate an effect of non-optimal positions and/or orientations on the pressure estimate. It is also possible that above-mentioned correction rule is a parametrized mathematical formula whose parameters are based on empirical test results of the kind mentioned above.

The non-limiting, specific examples provided in the description given above should not be construed as limiting the scope and/or the applicability of the appended claims. Furthermore, any list or group of examples presented in this document is not exhaustive unless otherwise explicitly stated.

1. A device for detecting motion of a surface, the device comprising:
a light source configured to emit light,
a focusing lens configured to focus the light, and
a detector configured to receive the focused light reflected off the surface and configured to detect motion of a distribution pattern of the received light, the motion of the distribution pattern of the received light being indicative of the motion of the surface reflecting the light,
wherein the focusing lens is an aspherical lens having a conic constant in a range from -1.5 to -0.5, and that a diameter of the focusing lens is at least 60% of a distance from a light egress surface of the focusing lens to a beam waist of the focused light.

2. The device according to claim 1, wherein the focusing lens is a plano-convex lens so that the light egress surface of the focusing lens has the conic constant in the range from -1.5 to -0.5 and a light ingress surface of the focusing lens is planar.

3. The device according to claim 1, wherein the focusing lens is a plano-convex lens so that a light ingress surface of the focusing lens has the conic constant in the range from -1.5 to -0.5 and the light egress surface of the focusing lens is planar.

4. The device according to claim 1, wherein the light egress surface of the focusing lens is non-planar and a light ingress surface of the focusing lens is non-planar so that a combined optical effect of the light ingress and light egress surfaces is a same as an optical effect of a plano-convex lens whose conic constant of a convex surface is in the range from -1.5 to -0.5.

5. The device according to claim 1, wherein the detector comprises an array of photosensor elements, changes of differences between output signals of the photosensor elements being indicative of the motion of the distribution pattern of the received light.

6. The device according to claim 1, wherein the device comprises a collector lens configured to direct, to the detector, the light reflected off the surface.

7. The device according to claim 1, wherein the conic constant is in a range from -1.3 to -0.6.

8. The device according to claim 1, wherein the conic constant is in a range from -1.1 to -0.7.

9. The device according to claim 1, wherein the conic constant is in a range from -0.9 to -0.7.

10. The device according to claim 1, wherein the diameter of the focusing lens is at least 70% of the distance from the light egress surface of the focusing lens to the beam waist of the focused light.

11. The device according to claim 1, wherein the diameter of the focusing lens is at least 90% of the distance from the light egress surface of the focusing lens to the beam waist of the focused light.

12. The device according to claim 1, wherein the diameter of the focusing lens is at least 110% of the distance from the light egress surface of the focusing lens to the beam waist of the focused light.

13. An apparatus for detecting pressure of an eye, the apparatus comprising:
an excitation source configured to direct airborne excitation to the eye to deform a surface of the eye,
a detector device configured to detect motion of the surface of the eye, and
a processing device configured to determine an estimate of the pressure of the eye based on the detected motion of the surface of the eye,
wherein the detector device comprises:
a light source configured to emit light,
a focusing lens configured to focus the light, and
a detector configured to receive the focused light reflected off the surface of the eye and configured to detect motion of a distribution pattern of the received light, the motion of the distribution pattern of the received light being indicative of the motion of the surface of the eye reflecting the light,
wherein the focusing lens is an aspherical lens having a conic constant in a range from -1.5 to -0.5, and that a diameter of the focusing lens is at least 60% of a distance from a light egress surface of the focusing lens to a beam waist of the focused light.

14. The apparatus according to claim 13, wherein the processing device is configured to measure time between a first time instant when the excitation source directs the airborne excitation to a first spot on the surface of the eye and a second time instant when the detector device detects the motion from a second spot on the surface of the eye, and to determine the estimate of the pressure of the eye based on the measured time.

15. The apparatus according to claim 13, wherein the processing device is configured to measure oscillation frequency related to the motion of the surface of the eye, and to determine the estimate of the pressure of the eye based on the measured oscillation frequency.

16. The apparatus according to claim 13, wherein the processing device is configured to correct the estimate of the pressure of the eye in accordance with a predetermined correction rule based on a location and/or a size of a light spot on a light receiving area of the detector, the location and/or the size of the light spot being indicative of a position and an orientation of the apparatus with respect to the eye.

17. The apparatus according to claim 13, wherein the apparatus further comprises a photosensor array configured to receive light reflected off the eye, and the processing device is configured to correct the estimate of the pressure of the eye in accordance with a predetermined rule based on a position of a pattern of the received light on the photosensor array.

18. A method for detecting motion of a surface, the method comprising:
emitting light,
focusing the light with a focusing lens so that the focused light is directed towards the surface, and
detecting motion of a distribution pattern of the focused light reflected off the surface, the motion of the distribution pattern of the reflected light being indicative of the motion of the surface,
wherein the focusing lens is an aspherical lens having a conic constant in a range from -1.5 to -0.5, and that a diameter of the focusing lens is at least 60% of a distance from a light egress surface of the focusing lens to a beam waist of the focused light.

19. The device according to claim 2, wherein the detector comprises an array of photosensor elements, changes of differences between output signals of the photosensor elements being indicative of the motion of the distribution pattern of the received light.

20. The device according to claim 3, wherein the detector comprises an array of photosensor elements, changes of differences between output signals of the photosensor elements being indicative of the motion of the distribution pattern of the received light.
